(19) United States
(12) Patent Application Publication
(10) Pub. No.: US 2007/0232867 A1
(43) Pub. Date: Oct. 4, 2007

(54) PROCESS AND SYSTEM FOR SETTING A PATIENT MONITOR

(75) Inventor: Hans-Ullrich HANSMANN, Barnitz (DE)

Correspondence Address: MCGLEW & TUTTLE, PC, P.O. BOX 9227, SCARBOROUGH STATION, SCARBOROUGH, NY, 10510-9227, US

(73) Assignee: DRAEGER MEDICAL AG & CO. KG, Luebeck (DE)

(21) Appl. No.: 11/612,693

(22) Filed: Dec. 19, 2006

(30) Foreign Application Priority Data
Apr. 1, 2006 (DE) 10 2006 015 291.3

(51) Int. Cl. A61B 5/00; G06F 19/00

(52) U.S. Cl. 600/300; 600/301; 702/19

(57) ABSTRACT

An automated process for setting a patient monitor (1) for detecting vital parameters of the patient measured by patient sensors as a function of the patient's current situation is provided. The process includes performing a statistical analysis from the measured values of the vital parameters in a computing unit for a plurality or all measured data of at least one of the measured vital parameters. The settings of the patient monitor (1) are adapted as a function of the statistical analysis performed, the adaptation of the settings pertaining at least to the vital parameters to be measured themselves, the frequency of measurements, the data quality and/or properties of the data transmission.

### CROSS REFERENCE TO RELATED APPLICATIONS

This application claims the benefit of priority under 35 U.S.C. § 119 of DE 10 2006 015 291.3 filed Apr. 1, 2006, the entire contents of which are incorporated herein by reference.

### FIELD OF THE INVENTION

The present invention pertains to a process for setting a patient monitor for detecting vital parameters of a patient measured by means of patient sensors as a function of the current situation of the patient.

### BACKGROUND OF THE INVENTION

Patients are commonly provided with a monitoring system within the clinical monitoring of intensive care units. Various vital parameters are usually measured with such systems, continuously or at least very frequently. Examples of measured vital parameters are Electrocardiograms (ECGs) signals, temperature, oxygen saturation, respiration rate, heart rate, non-invasive blood pressure, and pulse transit time.

A similar monitoring system is used sometimes outside the intensive care units or a system with electric patient data transmission is used.

Simple sensors with Global Mobile (Communications) System (GMS) (handy/mobile/cell) transmission are sometimes used outside hospitals in the area of outpatient care.

All these patient monitors transmit either continuously, as in intensive care units, or at a fixed measuring and transmission rate (telemetry) or upon individual requests of the user as event recorders.

This type of monitoring, which is known, in principle, is described, for example, in DE 103 45 171 A1, DE 198 49 607 C1 and DE 693 08 322 T2.

All prior-art patient monitoring systems have a fixed setting of the measured vital parameters including the quality of measurement, rate of transmission and limit values. Adaptation to changed ambient/situation conditions can be performed by a manual intervention only. As a result, there arise situations in which too few or unsuitable measured data are available for patients who run the risk of developing a crisis or the patients are stressed needlessly, the transmission network is loaded needlessly and/or the service life of the energy storage means is needlessly reduced in case of mobile patient monitor systems.

### SUMMARY OF THE INVENTION

The object of the present invention is to provide an automated process for setting a patient monitor for detecting vital parameters of a patient which are measured by means of patient sensors.

According to the invention, a process and system are provided for setting a patient monitor for detecting vital parameters of a patient that are measured by means of one or more patient sensors. The process includes performing a statistical analysis from the measured values of the vital parameters in a computing unit for a plurality or all measured data of at least one of the vital parameters. The settings of the patient monitor are then adapted depending on the statistical analysis performed. The adaptation of the settings pertains at least to the vital parameters to be measured themselves, the frequency of measurements, the data quality and/or properties of the data transmission.

An essential advantage of the process according to the invention is that the restriction for the patient due to the vital parameter monitoring is reduced to the adapted, necessary need. Furthermore, the amount of data to be transmitted is reduced by the process coordinated with the patient's status and the energy consumption is reduced. The patient-relevant rating criteria can be adapted due to the rated status of the patient and they may ensure a more stable alarm conditioning.

No additional staff is needed due to the automation of the process.

The patient data is measured by means of a monitoring system. This system comprises many individual patient monitors, also called patient units, with patient sensors and one or more central stations. The patient monitors are designed as small, portable monitors, which transmit the data into the hospital network or “TeleHealthCare” system in a wireless manner or via a fixed infrastructure, depending on the patient's mobility. For example, the Drager Infinity™ monitors are designed in a similar manner.

The receiving central station can feed information and settings back to the individual patient monitors. There are possibilities of bidirectional communication in terms of data systems engineering, but there also are audio and video transmission possibilities.

A meaningful and necessary data acquisition profile is calculated within the central station for every individual patient, i.e., rules are set up as to the conditions under which the time grid for requesting the measurement of a parameter shall be set up. The quality of a vital parameter measurement can also be varied in terms of resolution, averaging interval or number of leads (ECG). The bandwidth may range from the continuous detection and transmission of all vital parameters on-line to the replacement of the measurements by direct communication with the patient through a display, loudspeaker, microphone and by pressing a button.

These rules are based on an assessment of the patient by the medical staff and especially by the physician and may vary based on a trend observation or a statistical analysis of the history of the vital parameters. The change in the rules within one risk group can be calculated by a trend observation in respect to the variance of the measured values and the baseline value.

The rates of measurement may range from continuous to rare individual measurements. Individual measurements, which are within the expectation window, may possibly be evaluated and stored on the site only, without activating data transmission. In addition, it is possible to carry out measurements as a response to an individual request (“request”) by the central station or the patient.

A set of rules, which enables the medical staff to vary the settings for monitoring the patient's status within absolute or individual limits, is installed according to the present invention in the central station or in the patient monitor. It is possible to change both the limits for setting the patient monitor and the limits for the measured values, which shall lead to a change in the setting.

Similarly to the individual limit values of a conventional patient monitor, the limits are set for individual vital parameters. Depending on the patient's risk group, the clinical picture or other individual reasons, narrower limits may also be set by the medical staff within these limits. A set of settings, which can be used as a reference value for the setting for new patients, may be stored herefor.

The rules that are used for the evaluation are based on methods that are already used in other technical areas. Thus, a high percentage of the batches of new products is evaluated or measured statistically in quality assurance for acceptance. The percentage of parts to be qualified within a batch is reduced on the basis of the statistical distribution of the measured values (center and width of the gaussian distribution curve) and the increasing number of properties to be expected.

Control mechanisms are commonly installed in communications engineering and a counting unit is commonly actuated in each transmission unit. Disturbed transmissions are counted with a greater negative value and transmissions as expected are counted with a lower positive value.

Both methods are used to make possible an automatic adaptation under medical supervision. The statistical analysis of a blood pressure curve leads to a uniform curve in case of an unremarkable patient and could lead to a less frequent measurement in case of constant mean value and smaller spread. On the other hand, if a deviation is detected, it is also possible to respond rapidly and to increase the frequency of measurements in order to reliably detect the changes and to detect possible rapid gradients in the curve in time. The frequency of measurements may be reduced only after a preset number of “good” measured values, whereas “poor” measured values lead to an increase in the frequency of measurements more rapidly.

The rules that are used for the method may also be based on relationships between the different vital parameters. A change in one vital parameter may require more frequent measurement of another vital parameter in order to obtain a medically sufficient picture of the patient.

The frequency of a frequent blood pressure measurement, performed at 15-minute intervals, can be reduced in case of a low standard deviation and a mean value in the expectation window and possibly replaced by simpler measurement methods, such as pulse wave transit time, with limited information content. On the other hand, a detected increased blood pressure may also lead to more frequent ECG signal transmission.

The request to perform a measurement may also be generated directly from the central station. This makes it possible for the medical staff to detect a current status.

Besides the measured vital parameters, other information may be transmitted via the patient units as well.

This may include the administration of a drug (for example, “High Level User”), certain background information (for example, motion artifacts caused by rehabilitation procedures, walking), stress situation (patient) or a direct audio or video communication. Other additional measured values such as acceleration, position and ambient temperature, may be useful for a specific assessment or also be used for a plausibility check.

Based on localization information, for example, by means of the Radio Frequency Identification (RFID) technology, which is transmitted to the mobile patient units and to the central station through certain central passages, localization can be carried out at least in some areas even in case of interruption in communication.

The number of measurements carried out and the quality thereof are adapted by this process to the extent necessary for medical safety. The stress for the patient is correspondingly adapted as a result to the current individual situation. The medical safety of monitoring is preserved even under changing circumstances of the patient without needlessly frequent measurements having to be performed.

The adapted measurement mode leads to further advantages:

a) The power consumption of the patient unit, i.e., the patient monitor, with individual rates of measurement can be drastically reduced compared to a standardized general, reliable rate of measurement depending on the patient's current situation and the risk group to which the patient belongs. Mobile patients, whose independence and mobility are a decisive point in recovery, can move about markedly longer with the same battery capacity, independently from a charging station, or, as an alternative, they can be equipped with smaller, lighter-weight energy storage means.

b) The amount of measured data that is to be transmitted can be compressed to a high information content in case of a corresponding design of the set of request rules. This makes it possible to reduce the transmission times and consequently to reduce the power consumption and the rate of utilization of the transmission network.

c) The additional information, which is available about the patient, makes possible the specific analysis of the data in respect to the assessment of the patient's medical risk. Thus, a mobile patient may be subject to a physical stress that leads to a brief increase in the patient's pulse. Temporary shift of the alarm limits may ensure during an observed physical activity that no needless alarm will be generated. The reliability of the evaluation of the patient's status is improved and the alarm criteria are stabilized.

d) The localization of the area in which the patient is located is favorable for the speed with which assistance is offered. The additional information may also be useful in case of short-term redisposition within the facilities of the medical service provider, i.e., the hospital or TeleHealthCare system. The changed situation may be used to communicate the disposition to the patient in time.

e) The localization may be used to adapt the settings of the patient monitor to the conditions of the particular area when certain areas are entered. These include, e.g., the leaving of a building or the entry into a certain therapy room with certain applications and physical stress, change in position or temperature that are associated therewith. Provisions may also be made for temporarily shutting off the patient unit while the patient is in a restroom or the like.

f) The bilateral communications possibility expands the patient unit by direct address in both directions. Information, such as changed diagnosis dates, can be entered in a calendar by data systems engineering. However, audio or video transmissions may also be used to carry out short-term information or a personal balancing. A flexible information system can be obtained in conjunction with the localization of the area in which the patient is located.

g) It is possible due to the bilateral communication to give instructions to the patient. These instructions may be used to communicate certain behavioral guidelines in case of incorrect measurements. The avoiding of motion artifacts during the measurement or the announcement of certain measurements, such as an NIBP (non-invasive blood pressure) measurement with the blood pressure monitor cuff may be mentioned as examples.

h) If the measurement reveals an unacceptable stress for individual patients, the measurement may also be replaced temporarily by direct feedback by the patient. The desired freedom of movement can be achieved, especially in case of mobile patients, by the patient being able to report that he will be absent for a certain period of time for a walk or for visits. The expected measured values are replaced during this period by answering specific questions via the patient's patient unit later.

I) The central unit or alternatively the patient monitor may request a measurement. However, the measurement is carried out only after a trigger event by the patient.

An exemplary embodiment will be explained below on the basis of the Figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a patient monitor system for a patient; and

FIG. 2 is a schematic view showing the patient monitor in detail.

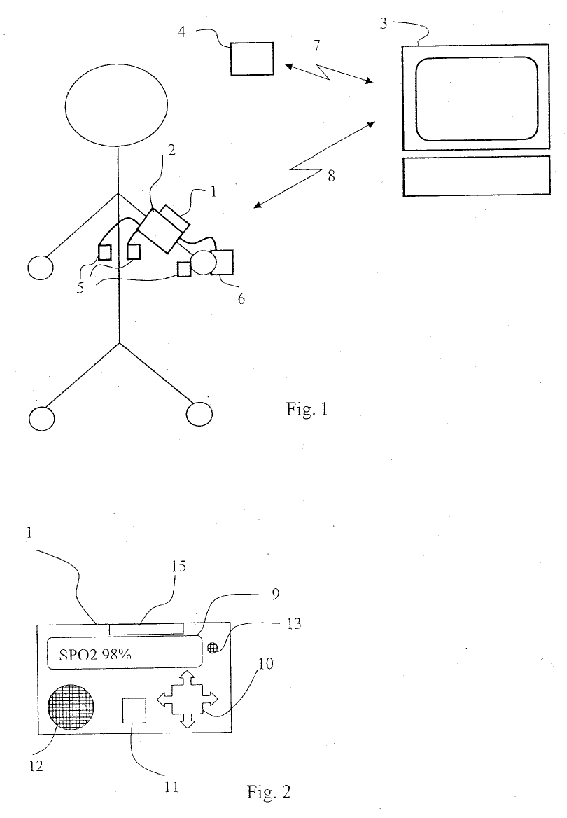

### DESCRIPTION OF THE PREFERRED EMBODIMENTS

Referring to the drawings in particular, the mobile patient monitor 1 with a plurality of patient sensors for the patient being shown is in communication connection with a central station 3. The patient monitor 1 is also called a patient unit.

The patient monitor 1 is equipped for measuring the vital parameters ECG (three electrodes 5 for two leads), an oscillatory blood pressure measurement or non-invasive blood pressure (NIBP) by means of an upper arm cuff 2 and for oxygen saturation measurement SPO₂ by means of a finger clip 6.

The patient monitor 1 is operated with a battery with an operating time of, e.g., 4 hours and transmits its data in a wireless manner by means of GSM-handy/mobile systems 8.

It is meaningful in this configuration for reasons of capacity to reduce the frequency of measurements as long as this is only associated with an acceptable loss of information.

The real-time ECG measurement requires a high data rate, at which it is necessary to maintain the data connection during the entire operating time (transmission mode). This means a shorter service life of the energy storage means (<4 hours) and represents a load for the transmission capacity of the transmitting/receiving units.

Even though the operation of the oxygen saturation sensor system does not require a high data rate, it does require a high output for the operation of the infrared diodes.

The upper arm cuff 2, which must be inflated for each new measured value, requires the energy needed for pumping for every individual procedure and represents a considerable restriction for the patient, because each measurement requires a phase of immobilization and a certain hold time. Furthermore, the sensation of pressure is unpleasant for the patient and may lead to nerve damage in case of very frequent measurements.

The patient monitor 1 is set individually by the attending physician for every individual parameter, which is appropriate for the risk group of the patient and the patient's specific case history.

The patient monitor 1 is initialized, for example, as follows:

| Vital parameters | Rate of measurement | Transmission |
|---|---|---|
| ECG | Every 5 minutes: store 10 sec | Every 10 minutes: |
| | signal | 10 sec signals, 2 leads |
| NIBP | Every 15 minutes: 1 | Every 30 minutes: |
| | measurement cycle | Diastolic + systolic |
| | | values |
| SPO₂ | Every 2 minutes: 20 sec | Every 10 minutes: |
| | measurement | 5 values + mean value |

The limit values for the SPO₂ measurement are 96%. If lower values are found, spontaneous control measurements are carried out. The patient receives a message to make a conclusion about the reliability of the measurement results on the basis of the patient's response (acknowledgment). The rate of measurement is increased for all three parameters.

| Vital parameters | Rate of measurement | Transmission |
|---|---|---|
| ECG | Continuous | Every minute: 10 sec signal, |
| | | 2 leads |
| NIBP | Every 2 minutes: 1 | Every 2 minutes: diastolic + |
| | measurement cycle | systolic values |
| SPO₂ | Continuous | Every minute: 20 values + |
| | | mean value |

If unstable values are subsequently measured or the values are outside, there will be a direct visit by the medical staff. If an SPO₂ value of 94% is measured, the value is confirmed by an additional control measurement and a visit is triggered.

If no declining but stable values that are to be expected are observed, the setting is changed after 2 hours as follows:

| Vital parameters | Rate of measurement | Transmission |
|---|---|---|
| ECG | Every 5 minutes: store 10 sec | Every 10 minutes: |
| | signal | 10 sec signal, 1 |
| | | leads |
| NIBP | Every 30 minutes: 1 | Every 30 minutes: |
| | measurement cycle | Diastolic + systolic |
| | | value |
| SPO₂ | Every 5 minutes: 20 sec | Every 10 minutes: |
| | measurement | 2 averaged values |

After further stable values that are to be expected, the setting is changed in a comparable manner. The rate of measurement can be reduced in the further course after 2 days to the extent that the upper arm cuff 2 can be removed and only two measurements are to be performed per day.

| Vital parameters | Rate of measurement | Transmission |
|---|---|---|
| ECG | None | None |
| NIBP | 2 per day | 2 per day: diastolic + systolic |
| | | values + heart rate |
| SPO₂ | None | None |

An additional motion sensor 11 at the patient monitor 1, FIG. 2, can recognize phases of sleep on the basis of the motion profile and move the NIBP measurement outside these phases of sleep as long as this is compatible with the time intervals.

Should the patient feel an uncertainty or suspect a cardiac event, the patient can carry out an additional measurement cycle at any time and reset the setting to the previous one. In addition, it is possible to get directly in touch with the medical care staff. A display 9, a loudspeaker 12, a microphone 13 and a multifunction button 10 are used for this.

Both units, i.e., the central station 3 and the patient monitor 1, store the current settings. The central station 3 is the master for possible changes.

Continuous monitoring of the patient's vital parameters is thus ensured and the patient is slowly relieved corresponding to the patient's health status.

In case of the increased setting, the patient must change the battery every 4 hours at the latest or connect the patient monitor 1 to a charging station for a half hour. The battery is sufficient for 2 days of operation in the last setting.

Detectors 4, which can recognize the proximity of the patient monitor 1 on the basis of an RFID (Radio Frequency Identification) (transponder) 15, are located at strategic points, such as passages, within the hospital premises. This information is transmitted via the central station to 3 the patient monitor 1, as is indicated by the double arrow 7.

Should an information packet not be able to be transmitted, the loss of information is recognizable in the central station 3, because the expected information does not enter during the time window. The patient monitor 1 operates with storage of the set values in its internal memory until the transmission is re-established.

The assessment of the measured data for the vital parameters is preferably performed in the computing unit of the patient monitor 1 or alternatively in the central station 3.

While specific embodiments of the invention have been shown and described in detail to illustrate the application of the principles of the invention, it will be understood that the invention may be embodied otherwise without departing from such principles.

What is claimed is:

1. A process for setting a patient monitor for detecting vital parameters of a patient that are measured by means of one or more patient sensors, the process comprising:
performing a statistical analysis from measured values of the vital parameters in a computing unit for a plurality or all measured data of at least one of the vital parameters; and
adapting the settings of the patient monitor depending on the statistical analysis performed, the adaptation of the settings pertaining at least to the vital parameters to be measured themselves, frequency of measurements, data quality and/or properties of data transmission.

2. A process in accordance with claim 1, wherein the statistical analysis includes a sliding mean value formation with trend analysis and gradient monitoring.

3. A process in accordance with claim 1, wherein the statistical analysis calculates separate trends for different time windows for different vital parameters.

4. A process in accordance with claim 1, wherein an evaluation of measured values is performed as a function of a preset therapy goal, so that measured values of vital parameters, which suggest a worsening of the patient's status after comparison with stored values, are weighted more heavily than measured values that indicate an improvement in the patient's status.

5. A process in accordance with claim 1, wherein the adaptation of the settings takes place according to rules that are stored in advance for use by a computing unit, the rules comprising, one or more of a rate of adaptation, limits for a change of settings, warning limits and alarm limits.

6. A process in accordance with claim 1, wherein selected analyses or sum of the statistical analyses are taken into account for the adaptation of the settings.

7. A process in accordance with claim 1, wherein additional information, especially patient data and/or data on the patient's current status including a patient's position and motion of the patient, are included in the statistical analysis of the measured data.

8. A process in accordance with claim 1, wherein verification of measured values is performed via the patient monitor by means of a voice communication.

9. A process in accordance with claim 1, wherein the patient monitor receives additional patient information by means of a motion sensor with acceleration and/or position measurement.

10. A process in accordance with claim 1, wherein additional actual information of said patient monitor including a charge status of an energy storage means is sent to a central station.

11. A process in accordance with claim 1, wherein the measured vital parameters comprise one or more of the following:
ECG leads; heart rate; temperature; oxygen saturation; respiration rate; pulse transit time; patient position; and motion of the patient.

12. A process in accordance with claim 1, wherein statistical values derived from measured values of the vital parameters are used for the statistical analysis.

13. A process in accordance with claim 1, wherein the adapted settings of said patient monitor pertains to frequency of measurements, data quality of measured values, resolution of the measured values, measuring sequence, data transmission and/or performance of the measurements.

14. A patient monitoring process comprising:
providing one or more patient sensors;
providing a patient monitor operatively connected to said one or more patient sensors for detecting vital parameters of a patient that are measured by said one or more patient sensors;
providing a computing unit operatively connected to said patient monitor;
sending measured value data from said patient monitor to said computing unit in a data transmission, said measured value data being based on measurement of one or more vital parameters using said one or more patient sensors;
performing a statistical analysis with said computing unit for at least a plurality or all of said measured value data; and
changing settings of said patient monitor based on said statistical analysis performed, wherein said settings comprise one or more of a setting of a vital parameters to be measured, a frequency of measurements, data quality of measurements and/or properties of the data transmission.

15. A process in accordance with claim 14, wherein the statistical analysis includes a sliding mean value formation with trend analysis and gradient monitoring and the change of the settings takes place according to rules that are stored in advance for use by the computing unit, the rules comprising, one or more of a rate of adaptation, limits for a change of settings, warning limits and alarm limits.

16. A process in accordance with claim 14, wherein a verification of the measured values is performed via said patient monitor by means of a voice communication established between said patient monitor to said computing unit.

17. A process in accordance with claim 14, wherein said measurement of one or more vital parameters using said one or more patient sensors comprise one or more of: measurement using ECG leads; measuring heart rate; measuring patient and/or ambient temperature; measuring oxygen saturation; measuring respiration rate; measuring pulse transit time; measuring patient position; and measuring patient motion.

18. A process in accordance with claim 1, wherein said settings of said patient monitor further comprise one or more of a frequency of measurements, data quality of the measured values, a resolution of the measured values, a measuring sequence, data transmission and/or a performance of the measurements.

19. A patient monitoring system comprising:
one or more patient sensors;
a patient monitor operatively connected to said one or more patient sensors for detecting vital parameters of a patient that are measured by said one or more patient sensors;
a computing unit operatively connected to said patient monitor, said patient monitor sending measured value data to said computing unit in a data transmission, said measured value data being based on measurement of one or more vital parameters using said sensors, said computing unit performing a statistical analysis for at least a plurality or all of said measured value data and changing settings of said patient monitor based on said statistical analysis performed, wherein said settings comprise one or more of a setting of vital parameters to be measured, a frequency of measurements, data quality of measurements and/or properties of the data transmission.

20. A system in accordance with claim 19, wherein said measurement of one or more vital parameters using said one or more patient sensors comprise one or more of: measurement using ECG leads; measuring heart rate; measuring patient and/or ambient temperature; measuring oxygen saturation; measuring respiration rate; measuring pulse transit time; measuring patient position; measuring patient motion and said settings of said patient monitor further comprise one or more of a frequency of measurements, data quality of the measured values, a resolution of the measured values, a measuring sequence, data transmission and/or a performance of the measurements.
